(19) United States
(12) Patent Application Publication
(10) Pub. No.: US 2021/0178191 A1
(43) Pub. Date: Jun. 17, 2021

(54) RADIOTHERAPY SYSTEM

(71) Applicant: Medical Intelligence Medizintechnik GmbH, Schwabmunchen (DE)

(72) Inventors: Michael Lewald, Schwabmünchen (DE); Thomas Pfitzmaier, Ustersbach (DE); Christian Berchtenbreiter, Mertingen (DE)

(21) Appl. No.: 17/250,266

(22) PCT Filed: Jun. 26, 2019
(86) PCT No.: PCT/EP2019/066981

§ 371 Date: Dec. 22, 2020

(30) Foreign Application Priority Data
Jun. 29, 2018 (DE) 20 2018 103 733.1

(51) Int. Cl. A61N 5/10; A61G 13/02

(52) U.S. Cl. CPC A61N 5/1049; A61G 13/02; A61G 2210/50; A61N 2005/1055; A61N 2005/1092; A61N 2005/1063

(57) ABSTRACT

The disclosure provides a radiotherapy system, comprising: a bed, for supporting the patient; and a bridge, comprising one or more rolling elements for supporting the bed and allowing the bed to be moved along a surface of the bridge. The one or more rolling elements are located at respective fixed positions in the bridge.

### TECHNICAL FIELD

Embodiments of the present disclosure relate to medical methods and apparatus, and particularly to a medical apparatus comprising a radiotherapy system, and corresponding methods.

### BACKGROUND

BackgroundRecent developments in the field of radiotherapy have focussed on integrating an imaging system with the therapeutic system. The goal is to provide real-time, or near real-time, feedback on the location of an anatomical feature within the patient (e.g. a tumour) such that a therapeutic radiation beam can be more accurately controlled to target that feature, or therapy can be halted if the radiation beam has become misdirected (for example).

One suggested approach is to combine a linear accelerator-based therapeutic system with a magnetic resonance imaging (MRI) system within a single apparatus, known as an MRI-Linac. Such apparatus is described in a number of earlier applications by the present Applicant, including U.S. patent application Ser. No. 12/704,944 (publication no 2011/0201918) and PCT publication no 2011/127947. In the systems described in these earlier applications, a patient can be imaged and treated substantially simultaneously while lying in the same position.

MRI systems comprise one or more coils for generating the strong magnetic fields on which the MRI process relies. For example, the system may comprise one or more coils for generating a primary magnetic field; and one or more coils for generating a gradient magnetic field that is superposed on the primary magnetic field and allows spatial encoding of the protons so that their position can be determined from the frequency at which resonance occurs (the Larmor frequency). In addition, an MRI system which is combined with a radiotherapy system may comprise one or more active shielding coils, which generate a magnetic field outside the MRI system of approximately equal magnitude and opposite polarity to the magnetic field generated by the primary magnetic coil. The more sensitive parts of the system (such as the radiation head) may be positioned in this region outside the coils where the magnetic field is cancelled, at least to a first order. The coils define a relatively narrow bore, in which the patient (or part of the patient) is placed during imaging.

Radiotherapy involves the delivery of highly energetic ionizing radiation to a target region within the patient (e.g. a tumour). The ionizing radiation damages or kills the cells in its path indiscriminately; healthy cells and tissues are damaged as well as non-healthy (e.g. cancerous) cells and tissue. Healthy cells are able to recover quicker than non-healthy cells, and this often leads to treatment being delivered in multiple, separate treatment sessions (known as “fractions”) to allow the healthy cells to recover in between treatment sessions. However, it is a general goal that radiation dosage to healthy cells should be minimized or avoided where possible, and thus the radiation beam should be shaped and delivered in an extremely accurate manner.

In order to achieve the accuracy required of radiotherapy, patients are often “set up” on the treatment bed or table by a trained clinician or therapist. This involves positioning the patient in a position which is most advantageous for delivering the radiation to the target without impacting surrounding healthy tissue. Various supports, restraints and radiation shields may be positioned on and around the patient to ensure that the patient is comfortable, that movement is minimized, and that the impact of any stray radiation on healthy tissue is reduced.

This presents a difficulty in combining a radiotherapy system with an MRI system, owing to the narrow bore of the MRI system. That is, conventional radiotherapy systems may have a relatively open area around the treatment table or bed, so that the patient can be set up in the same position as they will ultimately be treated in. However, that is not possible in MRI—radiotherapy systems, where the patient is positioned within a narrow bore during treatment. Thus the patient must be set up for treatment on a table or bed outside the bore (e.g. on a patient support), prior to that bed being transferred to the bore.

A system for transferring the patient from the patient support to the bore is therefore required.

### SUMMARY

Embodiments of the present disclosure seek to alleviate or overcome some or all of these problems.

Conventional MRI systems already comprise a system for transferring the patient into the bore for imaging purposes, of course. In these conventional systems, the bed or table is provided with rollers (e.g. wheels) which move in corresponding grooves or guides provided in the patient support and the bore of the MRI system. While this approach assists in reducing the friction between the bed and the patient support or bore, it presents difficulties when that MRI system is combined with a radiotherapy system.

It is beneficial for radiotherapy to be carried out in a consistent, predictable environment. The treatment is usually delivered in accordance with a treatment plan, which requires significant time and computing resources to generate. The treatment planning process is usually based on a planning image taken of the target region, where the target itself and surrounding sensitive structures (i.e. healthy organs and tissue which are particularly sensitive to radiation) are identified. The goal of treatment planning is to generate a treatment plan in which radiation dose to the target is maximized (e.g. is at least a defined minimum amount of radiation), radiation dose to the sensitive structures is minimized (e.g. is no more than a defined maximum amount of radiation), and radiation dose to healthy tissue is otherwise minimized or reduced to the extent possible. The treatment planning process may further take into account the limitations and abilities of the apparatus which is to deliver the treatment to the plan (e.g. available radiation power, radiation field size, etc). The treatment plan so generated (or the radiation dose profile achieved by the treatment plan) is output for review by a clinician, who may make changes or apply further constraints, requiring further iterations of the treatment planning process.

In order to reduce the complexities of the treatment planning process, it is beneficial if the background provided by the radiotherapy system is relatively uniform, or at least consistent from one treatment to the next. That is, ideally there should be no structural components which interfere with the beam or provide a source of stray radiation reflections. If such structural components are necessary, however, they should be consistent from one treatment to the next, such that the treatment planning process can take account of them more easily.

The problems with the conventional MRI system will now be apparent. The bed may be moved by different amounts into the bore, to account for different treatments, different target regions, and different patient anatomies. The rollers on the bed thus provide a structural component which varies inconsistently from one treatment to the next, and from one patient to the next.

In one aspect, the present disclosure provides a radiotherapy system, comprising: a bed, for supporting the patient; and a bridge, comprising one or more rolling elements for supporting the bed and allowing the bed to be moved along a surface of the bridge. The one or more rolling elements are located at respective fixed positions in the bridge.

### BRIEF DESCRIPTION OF THE DRAWINGS

For a better understanding of the present disclosure, and to show more clearly how it may be carried into effect, reference will now be made, by way of example, to the following drawings, in which:

FIG. 1 shows a side view of a combined radiotherapy and MRI system in cross-section according to embodiments of the present disclosure;

FIG. 2 is a plan drawing of an MRI bridge according to embodiments of the present disclosure;

FIG. 3 is a perspective view of one aspect of the MRI bridge according to embodiments of the present disclosure;

FIG. 4 shows a perspective cut-away view of a patient bed or table according to embodiments of the disclosure; and

FIG. 5 shows a perspective view of one aspect of the patient bed or table according to embodiments of the disclosure.

### DETAILED DESCRIPTION

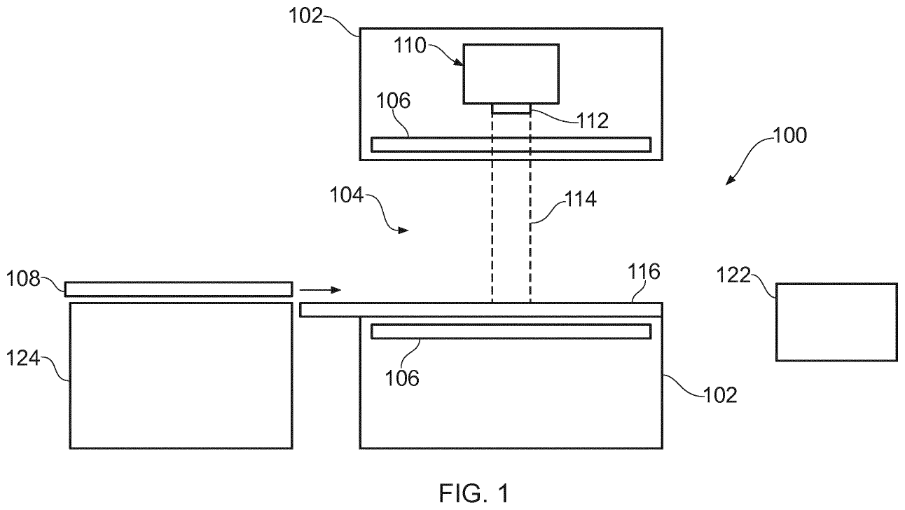

FIG. 1 is a schematic illustration of a combined radiotherapy and MRI system 100 according to embodiments of the present disclosure, showing a side view of the system in cross-section.

The system comprises a structure 102 defining a bore 104 in which a patient or part of a patient may be positioned during treatment. For example, the structure may comprise one or more coils 106 for generating a magnetic field as will be described in greater detail below. It will be noted that, as FIG. 1 shows the system 100 in cross-section, the structure 102 and the coils 106 are shown both above and below the bore 104.

A bed 108, for supporting the patient, can be positioned within the bore 104, and may be movable in a longitudinal direction into and out of the bore 108 to enable the patient to enter and exit the apparatus 100 before and after treatment. It will be understood that different terms may be used to describe the bed without departing from the scope of the claims appended hereto. For example, the bed 108 may also be called a table, or support. Use of the term “bed” herein does not imply that the corresponding apparatus must comprise a mattress or other cushioning, for example. The primary function of the bed is to support the patient and, as will be described below, move with the patient into the bore 104 of the MRI apparatus during use.

A patient support 124 may be provided to support the bed 108 outside the bore 104. In some embodiments, the patient support 124 may comprise one or more mechanisms for raising or lowering the bed 108 (when positioned on the support). For example, the height of the bed may be lowered to allow the patient to access or exit the bed 108 more easily before and after treatment. For treatment, the bed may be raised to a height at which the bed 108 can be transferred to the bore 104.

A suitable surface 116 is provided within the bore 104 to support the bed 108 while it is positioned within the bore. Such a surface may be known in the art as a bridge 116, and this term will be used hereinafter. The bridge 116 thus supports the bed 108 while it is located within the bore 104. The bridge 116 may also project outside the bore 104 to a limited extent, towards the patient support 124, such that the gap between the patient support 124 and the bridge 116 is relatively small, allowing the bed a smoother transition in its transfer from the support 124 to the bridge 116 and vice versa.

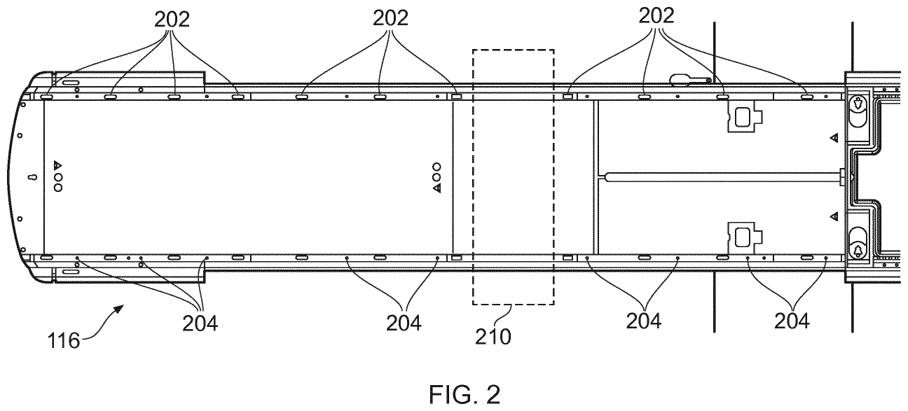

A low-friction surface (for example, one or more rolling elements such as rollers) and a driving mechanism (such as a driving piston, or a pulley) may be provided in the patient support 124 and/or the bridge 116 to enable such movement. This aspect will be discussed in more detail below, with respect to FIGS. 2 to 5.

The system 100 further comprises a radiotherapy apparatus which delivers doses of radiation to a patient supported by the bed 108. The radiotherapy apparatus comprises a radiation head 110 housing a source of radiation and a collimating device 112, which together generate a beam of therapeutic radiation 114. The source of radiation may take any suitable form (e.g. a radioactive source such as cobalt 60, a linear accelerator possibly in conjunction with an x-ray source, etc), and the beam may be formed of any suitable ionizing radiation, such as x-rays, electrons or protons (for example). The radiation will typically have an energy which is capable of having a therapeutic effect in a patient positioned on the bed 108. For example, a therapeutic x-ray beam may have an energy in excess of 1 MeV.

The collimating device 112 may be any device suitable for collimating the radiation beam to take a desired shape (for example, to conform to the shape of a target within the patient). In one embodiment, the collimating device may comprise a primary collimator and a second collimator. The primary collimator collimates the radiation to form a uniform beam shape (cone-, fan- and pyramid-shaped beams are known in the art), while the secondary collimator acts on the beam so collimated to adjust the shape to conform to the cross-sectional shape or a target within the patient, e.g. a tumour. In one embodiment, the secondary collimating device comprises a multi-leaf collimator, known to those skilled in the art. Such a device comprises one or more banks of elongate leaves (and typically comprises two such banks on opposite sides of the beam), with each leaf being individually moveable into and out of the radiation beam in order to block that part of the beam from reaching the patient. In combination, the leaves collectively act to shape the beam according to a desired cross-section.

The radiation head may be mounted on a chassis (not illustrated), and configured such that the radiation beam 114 is directed towards the patient. The chassis may be rotatable around an axis, with the point of intersection of the radiation beam with the axis being known as the “isocentre” of the apparatus. In this way, radiation can be directed towards a patient on the bed 108 from multiple directions, reducing the dose which is delivered to healthy tissue surrounding the target for treatment.

The system 100 further comprises an MRI apparatus, for producing images of a patient positioned on the bed 108. The MRI apparatus includes one or more magnetic coils 106 which act to generate a magnetic field for magnetic resonance imaging. That is, the magnetic field lines generated by operation of the magnetic coil 106 run substantially parallel to the central axis of the bore. The magnetic coils 106 may consist of one or more coils with an axis that runs parallel to, or is coincident with the axis of rotation of the chassis. The magnetic coils may be split into first and second magnetic coils, each having a common central axis, but separated by a window which is free of coils. In other embodiments, the magnetic coils 106 may simply be thin enough that they are substantially transparent to radiation of the wavelength generated by the radiation head 110. In yet further embodiments, the magnetic coils 106 may have a varying pitch, such that the pitch is relatively wide where the coils 106 intersect with the radiation beam 114, and relatively narrow in one or more regions outside the radiation beam 114. The magnetic coils may comprise one or more coils for generating a primary magnetic field; one or more coils for generating a gradient magnetic field that is superposed on the primary magnetic field and allows spatial encoding of the protons so that their position can be determined from the frequency at which resonance occurs (the Larmor frequency); and/or one or more active shielding coils, which generate a magnetic field outside the apparatus of approximately equal magnitude and opposite polarity to the magnetic field generated by the primary magnetic coil. The more sensitive parts of the system 100, such as the radiation head 110, may be positioned in this region outside the coils where the magnetic field is cancelled, at least to a first order.

The coils 106 may be arranged within the structure 102, which can additionally contain a system for keeping the coils cool (e.g. a cryogenic system based on liquid helium or similar).

The MRI system may further comprise an RF system (not illustrated) having an RF transmitter/receiver coil (also known as an imaging coil), which is brought close to the patient during imaging, transmits radio signals towards the patient, and detects the absorption at those frequencies so that the presence and location of protons in the patient can be determined. The RF system may include a single coil that both transmits the radio signals and receives the reflected signals, dedicated transmitting and receiving coils, or multi-element phased array coils, for example. The imaging coil may be affixed to the structure 102, or positioned close to the patient by a clinician.

In use, the MRI system can provide real-time imaging of a patient undergoing therapy, allowing accurate targeting of the treatment volume by the radiation beam 114 (for example through altered collimation by the collimating device 112), or automated shutdown if the patient moves significantly.

The system 100 further comprises a control apparatus 122, which is coupled to one or more components of the system 100 and controls their operation. The control apparatus will typically comprise a suitably programmed computing device (i.e. comprising processing circuitry configured to implement code stored in a computer-readable medium such as memory), but may also comprise dedicated electronic circuits.

The control apparatus 122 may be configured to control the operation of the radiotherapy parts of the system 100. For example, the apparatus 122 may control the source of radiation to generate a beam of therapeutic radiation having a particular energy, or comprising a particular radiation type; the apparatus 122 may control the collimator device 112 to conform the radiation beam 114 to a particular shape; the apparatus 122 may control the gantry, in order to rotate the radiation head 110 to one or more angles, such as a suitable angle for treatment; the apparatus may control movement of the bed 108 to a desired position for treatment of the patient.

The control apparatus 122 may also control the MRI parts of the system 100 so as to provide imaging information of the treatment volume of the patient. For example, the control apparatus 122 may control the magnetic coil 106 to generate a magnetic field of a certain strength, and a certain gradient; and the control apparatus 122 may control the imaging coil device to generate RF signals.

The control apparatus 122 may also control those parts of the system 100 relating to the position and movement of the bed 108. For example, the control apparatus 122 may control the patient support to take a particular height; the control apparatus 122 may control the driving mechanism controlling the movement of the bed 108.

FIG. 2 is a plan view of the bridge 116 according to embodiments of the disclosure.

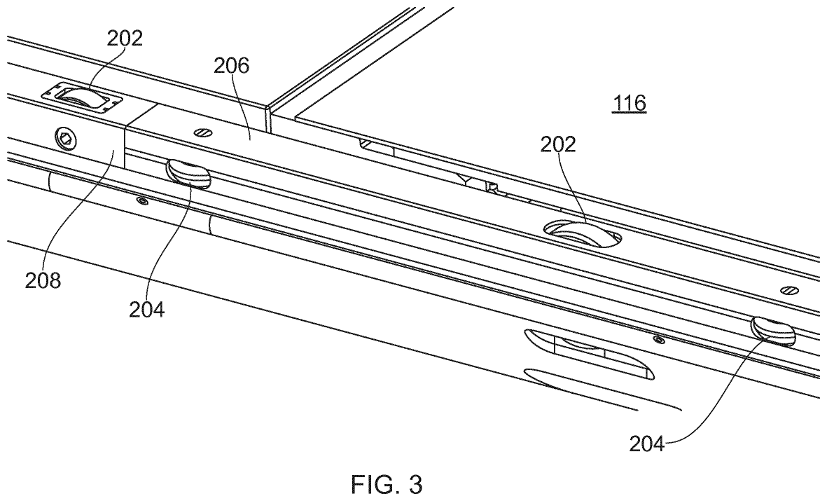

FIG. 3 shows one aspect of the bridge 116 in more detail.

According to embodiments of the disclosure, the bridge 116 comprises one or more rolling elements 202, 204. Note that this stands in contrast to conventional MRI systems, where rollers are provided in the bed and not the bridge. According to embodiments of the disclosure, rolling elements are provided in the bridge 116 (and potentially also the patient support 124, see below) but not in the bed 108. The bed 108 moves over the rolling elements 202, 204 (and in some cases is supported by them) in order to transport a patient into and out of the bore 104.

The rolling elements are provided in fixed positions within the bridge 116, and thus provide a consistent background for treatment planning purposes. FIG. 2 further shows a radiation window or volume 210 through which the radiation beam 114 can pass when the radiotherapy apparatus is active. Note that the radiation window 210 may define a volume between two parallel planes, to account for the different directions the radiation beam may be directed in when the gantry is rotatable. In the illustrated embodiment, the rolling elements 202, 204 are arranged outside the radiation window 210, so that the radiation beam 114 does not interact with the rolling elements and stray reflections of the radiation are reduced or minimized. In such embodiments, the rolling elements 202, 204 may be provided in positions which are adjacent to the radiation window 210, but not inside the radiation window 210, so that the bed 108 is firmly supported within the radiation window 210 (where accuracy of positioning is most important). In other embodiments, the rolling elements may be arranged within the radiation window 210 as well as outside the radiation window 210 (in which embodiments the fixed rolling elements still provide the benefit of a consistent background to the radiation).

FIG. 3 shows the arrangement of the rolling elements 202, 204 in more detail. It can be seen from the illustrated embodiment that first rolling elements 202 and second rolling elements 204 may be provided. The first rolling elements 202 are recessed into an upward-facing surface 206 of the bridge 116, but project from that surface to provide the low-friction surface on which the bed 108 will move. The first rolling elements 202 are thus positioned beneath, and bear the weight of, the bed 108 during use. The first rolling elements 202 may be rotatable around an axis that lies parallel to the upward-facing surface 206 (and to the main upward-facing surface of the bed 108), and perpendicular to the direction of motion of the bed 108.

The second rolling elements 204 are provided in a side-facing surface 208, which lies perpendicular to the upward-facing surface 206, such that the side-facing surface and the upward-facing surface together form a stepped profile. The second rolling elements 204 are also recessed into the side-facing surface 208, but project from that surface to provide a low-friction surface which acts to guide the bed 108 in its movement between the patient support 124 and the bridge 116. Thus, in contrast to the first rolling elements 202, which bear the weight of the bed 108 during use, the second rolling elements 204, together with the side-facing surface 208, act to guide the motion of the bed between the patient support 124 and the bridge 116. The second rolling elements 204 may be rotatable around an axis that lies parallel to the side-facing surface 208, and perpendicular to the direction of motion of the bed 108 and the upward-facing surface 206.

It is noted here that there is only one possible orientation of the patient support 124, the bridge 116 and the bed 108, in that the bed 108 must support a patient undergoing therapy, while the patient support 124 and the bridge 116 support the bed 108 (not necessarily at the same time). Thus there is a defined “upward” direction, which takes its conventional meaning with respect to gravity. Side-facing directions similarly have a conventional meaning, and can be defined relative to the upward direction (i.e. in that a side-facing direction may be defined as being transverse or, in some cases, perpendicular to the upward direction).

FIG. 2 shows the arrangement of rolling elements 202, 204 according to some embodiments. Thus the first rolling elements 202 may be arranged in pairs comprising one rolling element positioned at or near an edge of the bridge 116, and another rolling element positioned at a corresponding position at or near the opposing edge of the bridge 116. The bridge may comprise multiple such pairs of first rolling elements 202. Similarly, the second rolling elements 204 may be arranged in pairs comprising one rolling element positioned at or near an edge of the bridge 116, and another rolling element positioned at a corresponding position at or near the opposing edge of the bridge 116. The bridge may comprise multiple such pairs of second rolling elements 204.

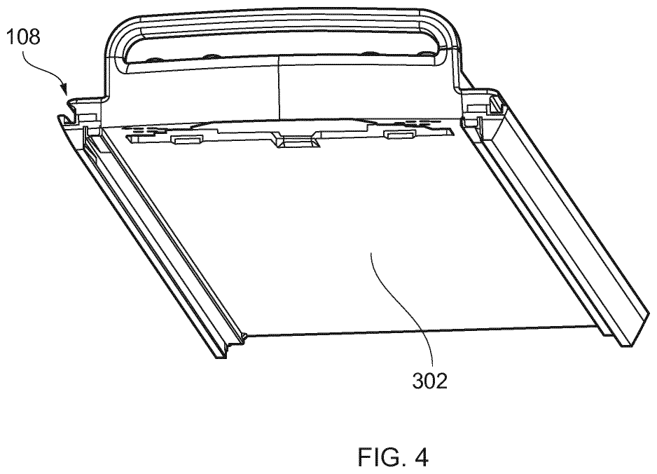

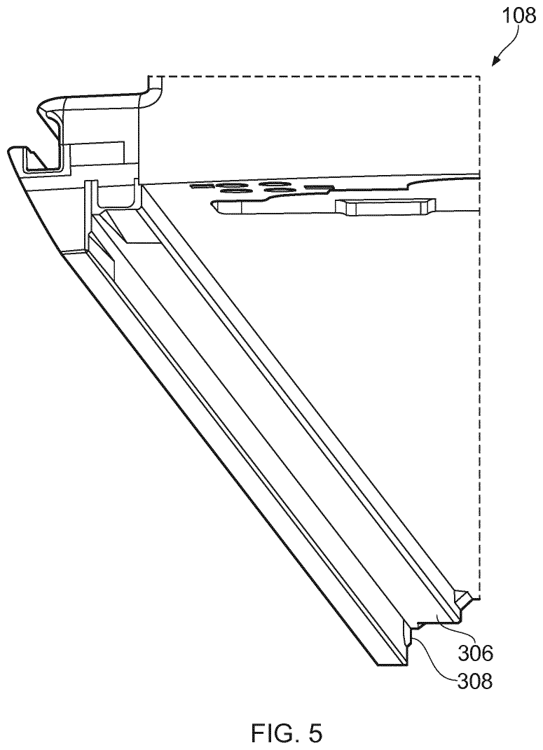

FIG. 4 shows, in cut-away cross section, a bed 108 according to embodiments of the disclosure. FIG. 5 shows an aspect of the bed 108 in greater detail. It will be understood by those skilled in the art that FIG. 5 shows only one side of the bed 108. A corresponding structure is provided on the opposing side of the bed 108.

It will be seen that the bed 108 comprises a stepped profile which is complementary to the stepped profile of the bridge 116 described above with respect to FIG. 3. Thus, the bed comprises a downward-facing surface 306, which engages with the first rolling elements 202 during use. In particular, as described above, the weight of the bed 108 is borne through the engagement of the downward-facing surface 306 with the first rolling elements 202. The bed further comprises an inward side-facing surface 308, which engages with the second rolling elements 204 during use. The interaction of the side-facing surface 208 with the second rolling elements 204 acts to guide the bed 108 in its motion between the patient support 124 and the bridge 116. In particular, the bed is permitted to move in a longitudinal direction, defining the movement of the bed 108 between the patient support 124 and the bridge 116, but is restricted by the interaction of the side-facing surface 308 with the second rolling elements 204 (and the interaction of a corresponding side-facing surface with second rolling elements on the opposing side of the bridge 116) from moving in a direction which is transverse or lateral to the longitudinal direction.

Different stepped profiles to those shown in FIGS. 3 and 5 may be provided without departing from the scope of the claims appended hereto. For example, alternative stepped profiles may comprise a groove or channel in the bed 108, within which the rolling elements of the bridge 116 or patient support 124 can be received.

It will further be noted from FIG. 4 that a central portion 302 of the bed 108 (on which the patient rests during use) has a substantially uniform thickness. Thus the bed 108 presents a substantially consistent object which can be accounted for (or neglected) in the treatment planning process.

The rolling elements may comprise rollers (as illustrated), or any other suitable rolling mechanism. For example, the rolling elements may comprise ball bearings held in a fixed position but allowed to rotate.

The above description has focussed on rolling elements 202, 204 provided in the bridge 116. However, it will be understood that similar arrangements of rolling elements may be provided in the patient support 124. Thus, the arrangement of rolling elements shown in FIGS. 2 and 3 may also be provided in the patient support 124, to allow easy movement of the bed 108 from the patient support and on to the patient support 124.

The present invention thus provides a radiotherapy system in which rolling elements are provided in the bridge, to facilitate movement of a bed within the system (e.g. between a patient support and the bridge).

Those skilled in the art will appreciate that various amendments and alterations can be made to the embodiments described above without departing from the scope of the invention as defined in the claims appended hereto.

1. A radiotherapy system, comprising:
a bed, for supporting the patient;
a source of therapeutic radiation for generating a beam of radiation; and a bridge, comprising one or more supporting rolling elements for supporting the bed and allowing the bed to be moved over the one or more supporting rolling elements and along a surface of the bridge, wherein the one or more supporting rolling elements are located at respective fixed positions in the bridge.

2. (canceled)

3. The radiotherapy system according to claim 1, wherein the bridge further comprises one or more guiding rolling elements configured to guide motion of the bed between a patient support and the bridge.

4. The radiotherapy system according to claim 9, wherein a surface of the bed comprises a stepped profile shaped to engage with the guiding rolling elements and guide the movement of the bed.

5. The radiotherapy system according to claim 4, wherein the bridge comprises a complementary stepped profile for engagement with the stepped profile of the bed.

6. The radiotherapy system according to claim 5, wherein the supporting rolling elements and the guiding rolling elements are provided in one or more surfaces of the stepped profile of the bridge.

7. The radiotherapy system according to claim 5, wherein the bed is moveable in a first direction and wherein the stepped profile is configured to restrict movement of the bed in a second direction transverse to the first direction.

8. The radiotherapy system according to claim 1, wherein the supporting rolling elements comprise one or more pairs of rolling elements, each pair comprising a first particular rolling element towards a first edge of the bridge and a second particular rolling element towards a second, opposing edge of the bridge.

9. The radiotherapy system according to claim 3, wherein the supporting rolling elements are configured to rotate around a first axis which is parallel to an upward-facing surface of the bridge, and wherein the bridge further comprises second guiding rolling elements are configured to rotate around a second axis which is perpendicular to the upward-facing surface of the bridge.

10. (canceled)

11. (canceled)

12. The radiotherapy system according to claim 1, further comprising an MRI apparatus, and wherein the bridge is positioned within a bore of the MRI apparatus.

13. The radiotherapy system according to claim 1, wherein the supporting rolling elements comprise rollers.

14. The radiotherapy system according to claim 1, wherein the bed has a uniform thickness.

15. The radiotherapy system according to claim 1, wherein the supporting rolling elements are located at respective fixed positions to provide a consistent background for treatment planning.

16. The radiotherapy system according to claim 1, wherein the beam of radiation passes through a radiation window, and wherein the respective fixed positions are arranged outside of the radiation window.

17. The radiotherapy system according to claim 16, wherein the respective fixed positions are adjacent to the radiation window.

18. The radiotherapy system according to claim 3, wherein the one or more guiding rolling elements are located at respective fixed positions in the bridge to provide a consistent background for treatment planning purposes.

19. The radiotherapy system according to claim 3, wherein the beam of radiation passes through a radiation window, and wherein the one or more guiding rolling elements are located at respective fixed positions in the bridge outside the radiation window.

20. The radiotherapy system according to claim 6, wherein:
the supporting rolling elements are located in an upward-facing surface of the complementary stepped profile and the first axis is parallel to the upward-facing surface of the complementary stepped profile, and
the second rolling elements are located in a side-facing surface of the complementary stepped profile and the second axis is perpendicular to the upward-facing surface of the complementary stepped profile.

21. The radiotherapy system according to claim 3, wherein the guiding rolling elements comprise one or more pairs of rolling elements, each pair comprising a first particular rolling element towards a first edge of the bridge and a second particular rolling element towards a second, opposing edge of the bridge.

22. A system comprising the radiotherapy system of claim 1, further comprising a treatment planning apparatus configured to generate a radiotherapy treatment plan based on the one or more supporting rolling elements being in the fixed positions.

23. The system of claim 22, wherein the bed in the radiotherapy system has a uniform thickness and the treatment planning apparatus is further configured to generate the radiotherapy treatment plan based on the consistent background provided by the bed.
